(19) United States
(12) Patent Application Publication
(10) Pub. No.: US 2023/0404747 A1
(43) Pub. Date: Dec. 21, 2023

(54) VOICE GENERATION SYSTEM AND METHOD

(71) Applicant: Laronix Pty Ltd, Queensland (AU)

(72) Inventor: Farzaneh Ahmadi, Queensland (AU)

(73) Assignee: Laronix Pty Ltd, Queensland (AU)

(21) Appl. No.: 18/035,102

(22) PCT Filed: Nov. 4, 2021
(86) PCT No.: PCT/AU2021/051302

§ 371 Date: May 2, 2023

(30) Foreign Application Priority Data
Nov. 4, 2020 (AU) 2020904008

(51) Int. Cl. A61F 2/20; A61F 2/48; G10L 21/034; G10L 21/0208; G10L 21/0364

(52) U.S. Cl. CPC A61F 2/203; A61F 2/482; A61F 2/484; G10L 21/034; G10L 21/0208; G10L 21/0364; A61F 2002/206

(57) ABSTRACT

Described is a voice generation system. The voice generation system comprises a housing comprising a first opening and a second opening. The housing defines an air passage between the first opening and the second opening. The voice generation system comprises a moveable member located within the housing. The moveable member is configured to vibrate in response to air flowing in the air passage. The voice generation system comprises a transducer module configured to convert vibrations of the moveable member into an electrical signal. The voice generation system comprises a speaker module configured to convert the electrical signal into sound and to output sound into the oral cavity of a user. The voice generation system is configured such that during use, the air pressure at or near the second opening corresponds to an air pressure in the oral cavity of the user.

### TECHNICAL FIELD

The present invention generally relates to artificial voice generation. In further specific examples, the present invention relates to methods and systems for generating a voice.

### BACKGROUND

The larynx, also known as the voice box, is the organ used to generate the sound that humans use for speech. The larynx houses the vocal folds which are the source of a person's voice. When a healthy person speaks, the sound (or “the voice”) produced by the vocal folds in the larynx enters the vocal tract where the voice is filtered (e.g. by controlling movement of the tongue and lips) to produce speech. A person whose larynx has been surgically removed (through a laryngectomy) or bypassed (through a tracheostomy) may still be capable of controlling the vocal tract, but lacks the vocal folds to generate a voice. Such a person is therefore unable or inhibited in his ability to generate a voice without artificial aid.

One example artificial aid is an electrolarynx, which is a handheld device that a laryngectomy patient presses against the skin of his or her neck or face to speak. The device functions by inducing vibrations into the vocal tract as an artificial voice source that the person can then shape into speech by controlling movement of the tongue and lips. The voice produced by an electrolarynx, however, tends to have a robotic tone, as well as being generally inconvenient for the person to be forced to manually operate the device while speaking.

A tracheoesophageal voice prosthesis (TEP) is another type of voice prosthesis (artificial aid) conventionally employed by laryngectomy patients. During a laryngectomy, a permanent opening known as a stoma is produced in the neck of the patient for breathing through. As a result, the patient's trachea is no longer in communication with the vocal tract so that air from the lungs exits through the stoma and cannot enter the vocal tract. A TEP is a plastic valve which is surgically inserted inside the throat between the trachea and the oesophagus. The TEP allows air from the lungs to re-enter the oesophagus and, from there, travel through the throat and vibrate tissues inside the throat, thus generating a voice (similarly to how sound is generated during belching). While the resulting speech is intelligible, the TEP is a primitive solution and suffers from several critical drawbacks. For example, the TEP is highly invasive, it causes infection and swallowing bio-hazards, and the voice generated is limited to a hoarse and whispery quality.

There is a need for new or improved systems and/or methods for generating a voice or for generating an airflow through the vocal tract of a person.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

According to an example aspect, there is provided a voice generation system comprising: a housing comprising a first opening and a second opening, the housing defining an air passage between the first opening and the second opening; a moveable member located within the housing and configured to vibrate in response to air flowing in the air passage; a transducer module configured to convert vibrations of the moveable member into an electrical signal; and a speaker module configured to convert the electrical signal into sound and to output sound into the oral cavity of a user; wherein, the voice generation system is configured such that during use, the air pressure at or near the second opening corresponds to an air pressure in the oral cavity of the user.

In certain embodiments, the air flowing in the air passage is due to an air pressure difference between the first opening and the second opening.

In certain embodiments, the air pressure at or near the first opening corresponds to an air pressure at the neck of the user.

In certain embodiments, the air pressure at or near the first opening corresponds to an air pressure at the neck stoma of the user.

In certain embodiments, the first opening is configured to be in communication with a neck stoma of the user such that the air pressure at the first opening corresponds to the air pressure at the neck stoma of the user.

In certain embodiments, the second opening is configured to be in communication with the oral cavity of the user such that the air pressure at the second opening corresponds to the air pressure at the oral cavity of the user.

In certain embodiments, the voice generation system further comprises a tube having a first end connected to the housing around the second opening, wherein a second end of the tube is configured to be inserted into the oral cavity of the user.

In certain embodiments, the housing and the tube together define an air passage between the first opening of the housing and the second end of the tube.

In certain embodiments, the speaker module is located within the housing, and wherein the speaker module is configured to output the sound through the second opening.

In certain embodiments, the housing is configured to connect to an airflow source, the airflow source being configured to generate an airflow and to output the airflow into the oral cavity of the user.

In certain embodiments, the airflow source is a neck stoma of the user and the airflow is a respiratory airflow outputted from the user through the neck stoma.

In certain embodiments, the airflow source is an air pump.

In certain embodiments, the voice generation system further comprises: a pressure sensing module configured to attach to the neck of the user and to sense an air pressure at a neck stoma of the user; an air pump located within the housing and configured to generate an airflow that moves along the air passage from the first opening to the second opening;

and a controller configured to control the air pump based on the sensed air pressure at the neck stoma.

In certain embodiments, the air flowing in the air passage is due to a difference in the air pressure in the air passage and the air pressure at the second opening.

In certain embodiments, the second opening is configured to be in communication with the oral cavity of the user.

In certain embodiments, the voice generation system further comprises a tube having a first end connected to the housing around the second opening, wherein a second end of the tube is configured to be inserted into the oral cavity of the user.

In certain embodiments, the housing and the tube together define an air passage between the first opening of the housing and the second end of the tube.

In certain embodiments, the speaker module is located within the housing, and wherein the speaker module is configured to output the sound through the second opening.

In certain embodiments, the air pump is configured to generate an air pressure in the air passage corresponding to the sensed air pressure at the neck stoma.

In certain embodiments, the housing is configured to be secured to an auricle of the user.

In certain embodiments, the voice generation system is configured to be used hands free.

According to another example aspect, there is provided a voice generation system comprising: a housing comprising a first opening and a second opening, the housing defining an air passage between the first opening and the second opening, wherein the first opening is configured to be in communication with a neck stoma of the user; a moveable member located within the housing and configured to vibrate in response to air flowing in the air passage; a regulator located within the housing and configured to control a flow of air in the air passage; a transducer module configured to convert vibrations of the moveable member into an electrical signal; a speaker module configured to convert the electrical signal generated by the transducer module into sound and to output sound into the oral cavity of a user; an air pump configured to generate an airflow into the oral cavity of the user; a pressure sensing module configured to sense an air pressure in the oral cavity of the user; and a controller configured to control the regulator based on the sensed air pressure in the oral cavity.

In certain embodiments, the housing is configured to attach to the neck of a user such that the first opening is in connection with the neck stoma.

In certain embodiments, the housing is fixed to a neck harness configured to fit around the rear neck of the user.

In certain embodiments, the voice generation system further comprises: a flow sensing module located within the housing and configured to sense an airflow in the air passage; and

a second controller configured to control the air pump based on the sensed airflow in the air passage; wherein the air pump is configured to generate an airflow into the oral cavity corresponding to the sensed airflow in the air passage.

In certain embodiments, the air pump and the speaker module are located within a second housing which is configured to be secured to an auricle of the user, wherein the second housing comprises an opening configured to be in communication with the oral cavity of the user, and wherein the speaker module is configured to output the sound through the opening of the second housing.

In certain embodiments, the voice generation system further comprises a tube having a first end connected to the opening of the second housing, wherein a second end of the tube is configured to be inserted into the oral cavity of the user, and wherein the pressure sensing module is fixed to the second end of the tube.

In certain embodiments, the air pump, the speaker module, and the pressure sensing module are fixed to a denture unit configured to be secured to the oral cavity of the user.

In certain embodiments, the regulator is a further air pump that is configured to generate an air pressure in the air passage corresponding to the sensed air pressure in the oral cavity.

In certain embodiments, the regulator is an air valve configured to control the flow of air through the second opening.

In certain embodiments, the pressure sensing module is in wireless communication with the controller, and wherein the transducer module is in wireless communication with the speaker module.

In certain embodiments, the voice generation system further comprises a processing system.

In certain embodiments, the processing system is configured to reduce sound interference received at the transducer module.

In certain embodiments, the processing system is configured to reduce sound interference based on at least one electrical signal received from:

the transducer module configured to convert vibrations of the moveable member, and/or

at least one interference transducer module for receiving sound interference

In certain embodiments, the sound interference at the transducer module comprises sound outputted by the speaker module, and/or speech that is generated when sound outputted by the speaker module is modulated by movement of the user's mouth.

According to another example aspect, there is provided a voice generation system comprising: a housing comprising a first opening and a second opening, the housing defining an air passage between the first opening and the second opening; a moveable member located within the housing and configured to vibrate in response to air flowing in the air passage; a transducer module configured to convert vibrations of the moveable member into an electrical signal; a speaker module configured to convert the electrical signal into sound and to output sound into the oral cavity of a user; and a processing system configured to reduce sound interference received at the transducer module.

In certain embodiments, the processing system is configured to reduce sound interference based on at least one electrical signal received from:

the transducer module configured to convert vibrations of the moveable member, and/or

at least one interference transducer module for receiving sound interference

In certain embodiments, the processing system is configured to reduce sound interference by performing feedforward suppression.

In certain embodiments, feedforward suppression comprises one or more of: gain reduction, frequency selective noise reduction, notch filter, phase modulation, and frequency shifting.

In certain embodiments, the processing system is configured to reduce sound interference by performing adaptive feedback cancellation or residual feedback suppression.

In certain embodiments, adaptive feedback cancellation comprises adaptive filtering.

In certain embodiments, adaptive filtering is done with an FIR filter.

In certain embodiments, adaptive filtering is done using a (real-time) normalized least-mean-square (NLMS) algorithm.

In certain embodiments, the voice generation system further comprises at least one interference transducer module for receiving sound interference.

In certain embodiments, the sound interference at the transducer module comprises sound outputted by the speaker module, and/or speech that is generated when sound outputted by the speaker module is modulated by movement of the user's mouth.

In certain embodiments, the voice generation system further comprises a first interference transducer module configured to convert sound outputted by the speaker module into an electrical signal as an input for the processing system.

In certain embodiments, the voice generation system further comprises a second interference transducer module configured to convert speech that is generated when sound outputted by the speaker module is modulated by movement of the user's mouth into an electrical signal as an input for the processing system.

In certain embodiments, the processing system outputs at least one electrical signal to reduce sound interference.

In certain embodiments, the interference transducer module comprises any one of:

a microphone

a piezoelectric transducer

a magnetic pickup transducer,

an accelerometer

a voice sensor

a vibration sensor

In certain embodiments, the processing system is configured to process the electrical signal generated by the transducer module.

In certain embodiments, the processing system comprises hardware and software configured to improve a volume or quality of a voice encoded by the electrical signal.

In certain embodiments, the processing system comprises an AI voice conversion software to improve the quality of the voice encoded by the electrical signal.

In certain embodiments, the transducer module comprises a microphone.

In certain embodiments, the transducer module comprises a piezoelectric transducer.

In certain embodiments, the transducer module comprises a magnetic pickup transducer.

In certain embodiments, the speaker module comprises a loudspeaker array.

In certain embodiments, the transducer module comprises any one of:

a pressure sensor

a sound sensor

a vibration detection sensor

an accelerometer

In certain embodiments, the moveable member comprises a membrane.

In certain embodiments, the moveable member comprises multiple membranes with different levels of complexity.

According to another example aspect, there is provided a voice generation system comprising: a housing comprising a first opening and a second opening, the housing defining an air passage between the first opening and the second opening; a moveable member located within the housing and configured to vibrate in response to air flowing in the air passage; a transducer module configured to convert vibrations of the moveable member into an electrical signal; and a speaker module configured to convert the electrical signal into sound and to output sound into the oral cavity of a user; wherein, output sound is based on air pressure in the oral cavity of the user.

In certain embodiments, the voice generation system is configured such that during use, the air pressure at or near the second opening corresponds to an air pressure in the oral cavity of the user.

According to another example aspect, there is provided a method of generating a voice, comprising: providing a moveable member in communication with an airflow corresponding to a respiratory airflow of a user; detecting vibrations of the moveable member in response to the airflow; generating, using one or more electrical speakers, a sound derived from the detected vibrations; and supplying an airflow and the sound into the oral cavity of the user.

In certain embodiments, the moveable member is provided in an air channel, the air channel being subject to a first air pressure at a first end of the air channel and a second air pressure at a second end of the air channel.

In certain embodiments, the second air pressure corresponds to an air pressure in the oral cavity of the user.

In certain embodiments, the first air pressure corresponds to an air pressure of a neck stoma of the user.

In certain embodiments, the method further comprises:

sensing the air pressure and/or an airflow at the neck stoma of the user; and

generating, using an air pump, the first air pressure and the airflow of the neck stoma.

In certain embodiments, the method further comprises:

sensing the air pressure in the oral cavity of the user; and

generating, using an airflow control element, the second air pressure.

In certain embodiments, the airflow control element is an air pump.

In certain embodiments, the airflow control element is an air valve.

In certain embodiments, the airflow control element is any one of:

a suction

an actuator

In certain embodiments, the method further comprises channelling, within an air passage, a respiratory airflow of the user, wherein the airflow is the channelled respiratory airflow.

In certain embodiments, the method further comprises: converting, using a transducer, the vibrations of the moveable member into an electrical signal; processing the electrical signal; and supplying the processed electrical signal to the one or more electrical speakers for generating the sound.

In certain embodiments, processing the electrical signal comprises improving a volume or quality of a voice encoded by the electrical signal.

In certain embodiments, the method further comprises reducing sound interference generated by the one or more electrical speakers.

According to an example aspect, there is provided a voice generation system comprising: a housing comprising a first opening and a second opening, the housing defining an air passage between the first opening and the second opening; a moveable member located within the housing and configured to vibrate in response to air flowing in the air passage; a transducer module configured to convert vibrations (or sound produced by the vibrations) of the moveable member into an electrical signal; and a speaker module configured to convert the electrical signal into sound and to output the sound into the oral cavity of a user.

In certain embodiments, the air flowing in the air passage is due to an air pressure difference between the first opening and the second opening.

In certain embodiments, the first opening is configured to be in communication with a neck stoma of the user.

In certain embodiments, the housing is configured to attach to the neck or chest of the user such that the first opening is in connection with the neck stoma.

In certain embodiments, the second opening is configured to be in communication with the oral cavity of the user.

In certain embodiments, the voice generation system further comprises a tube having a first end connected to the housing around the second opening, wherein a second end of the tube is configured to be inserted into the oral cavity of the user.

In certain embodiments, the housing and the tube together define an air passage between the first opening of the housing and the second end of the tube.

In certain embodiments, the speaker module is located within the housing, and the speaker module is configured to output the sound (i.e. the voice) through the second opening.

In certain embodiments, the housing is configured to connect to an airflow source, the airflow source being configured to generate an airflow and to output the airflow into the oral cavity of the user.

In certain embodiments, the airflow source is a neck stoma of the user and the airflow is a respiratory airflow outputted from the user through the neck stoma.

In certain embodiments, the airflow source is an air pump.

In certain embodiments, the voice generation system further comprises: a pressure sensing module configured to attach to the neck of the user and to sense an air pressure at a neck stoma of the user; an air pump located within the housing and configured to generate an airflow that moves along the air passage from the first opening to the second opening; and a controller configured to control the air pump based on the sensed air pressure of the neck stoma (as monitored by the pressure sensing module).

In certain embodiments, the air flowing in the air passage is due to a difference in the air pressure in the air passage and the air pressure at the second opening.

In certain embodiments, the second opening is configured to be in communication with the oral cavity of the user.

In certain embodiments, the voice generation system further comprises a tube having a first end connected to the housing around the second opening, wherein a second end of the tube is configured to be inserted into the oral cavity of the user.

In certain embodiments, the housing and the tube together define an air passage between the first opening of the housing and the second end of the tube.

In certain embodiments, the speaker module is located within the housing, and the speaker module is configured to output the sound through the second opening.

In certain embodiments, the air pump is configured to generate an air pressure in the air passage corresponding to the sensed air pressure at the neck stoma.

In certain embodiments, the housing is configured to be secured to an auricle of the user.

According to another example aspect, there is provided a voice generation system comprising: a housing comprising a first opening and a second opening, the housing defining an air passage between the first opening and the second opening, wherein the first opening is configured to be in communication with a neck stoma of the user; a moveable member located within the housing and configured to vibrate in response to air flowing in the air passage; a transducer module configured to convert vibrations of the moveable member into an electrical signal; a speaker module configured to convert the electrical signal into sound and to output the sound into the oral cavity of a user; a first air pump located within the housing and configured to generate an air pressure in the air passage at the second opening; a pressure sensing module configured to sense an air pressure in the oral cavity of the user; and a controller configured to control the first air pump based on the sensed air pressure of the oral cavity; an airflow sensing module configured to sense the airflow of the stoma of the user; a second air pump configured to generate an airflow into the oral cavity of the user based on the sensed air flow of the neck stoma.

According to another example aspect, there is provided a voice generation system comprising: a housing comprising a first opening and a second opening, the housing defining an air passage between the first opening and the second opening, wherein the first opening is configured to be in communication with a neck stoma of the user; a moveable member located within the housing and configured to vibrate in response to air flowing in the air passage; a regulator located within the housing and configured to control a flow of air in the air passage; a transducer module configured to convert vibrations of the moveable member into an electrical signal; a speaker module configured to convert the electrical signal generated by the transducer module into sound and to output the sound into the oral cavity of a user; an air pump configured to generate an airflow into the oral cavity of the user; a pressure sensing module configured to sense an air pressure in the oral cavity of the user; and a controller configured to control the regulator based on the sensed air pressure in the oral cavity.

In certain embodiments, the housing is configured to attach to the neck of a user such that the first opening is in connection with the neck stoma.

In certain embodiments, the housing is fixed to a neck harness configured to fit around the rear neck of the user.

In certain embodiments, the voice generation system further comprises: a flow sensing module located within the housing and configured to sense an airflow in the air passage (e.g. an airflow of the stoma); and a second controller configured to control the air pump based on the sensed airflow in the air passage; wherein the air pump is configured to generate an airflow into the oral cavity corresponding to the sensed airflow in the air passage.

In certain embodiments, the air pump and the speaker module are located within a second housing which is configured to be secured to an auricle of the user, wherein the second housing comprises an opening configured to be in communication with the oral cavity of the user, and wherein the speaker module is configured to output the sound through the opening of the second housing.

In certain embodiments, the voice generation system further comprises a tube having a first end connected to the opening of the second housing, wherein a second end of the tube is configured to be inserted into the oral cavity of the user, and wherein the pressure sensing module is fixed to the second end of the tube.

In certain embodiments, the air pump, the speaker module, and the pressure sensing module are fixed to a denture unit configured to be secured to the oral cavity of the user.

In certain embodiments, the regulator is a further air pump that is configured to generate an air pressure in the air passage corresponding to the sensed air pressure in the oral cavity. In certain embodiments, the regulator is an air valve configured to control the flow of air through the second opening.

In certain embodiments, the pressure sensing module is in wireless communication with the controller, and wherein the transducer module is in wireless communication with the speaker module.

In certain embodiments, the voice generation system further comprises a processing system configured to process the electrical signal generated by the transducer module.

In certain embodiments, the processing system comprises hardware and software configured to improve a volume or quality of a voice encoded by the electrical signal.

In certain embodiments, the processing system comprises an artificial intelligence (AI) voice conversion software to improve the quality of the voice encoded by the electrical signal.

In certain embodiments, the software is implemented as a trainable artificial intelligence (AI) algorithm.

In certain embodiments, the transducer module comprises a microphone.

In certain embodiments, the transducer module comprises a piezoelectric transducer.

In certain embodiments, the transducer module comprises a magnetic pickup transducer.

In certain embodiments, the speaker module comprises a microspeaker or a loudspeaker array.

In certain embodiments, the moveable member comprises a membrane.

In certain embodiments, the moveable member comprises multiple membranes with different levels of complexity.

In certain embodiments, the moveable member is a silicone model of vocal folds.

According to another example aspect, there is provided a method of generating a voice, comprising: providing a moveable member in communication with an airflow corresponding to a respiratory airflow of a user; detecting vibrations of the moveable member in response to the airflow; generating, using one or more electrical speakers, a sound derived from the detected vibrations; and supplying an airflow and the sound into the oral cavity of the user.

In certain embodiments, the moveable member is provided in an air channel, the air channel being subject to a first air pressure at a first end of the air channel and a second air pressure at a second end of the air channel.

In certain embodiments, the first air pressure corresponds to an air pressure of a neck stoma of the user, and wherein the second air pressure corresponds to an air pressure in the oral cavity of the user.

In certain embodiments, the method further comprises: sensing the air pressure and/or an airflow at the neck stoma of the user; and generating, using an air pump, the first air pressure and the airflow of the neck stoma.

In certain embodiments, the method further comprises: sensing the air pressure in the oral cavity of the user; and generating, using a regulator or an airflow control element, the second air pressure. In certain embodiments, the airflow control element is an air pump. In certain embodiments, the airflow control element is an air valve.

In certain embodiments, the method further comprises channelling, within an air passage, a respiratory airflow of the user, wherein the airflow is the channelled respiratory airflow.

In certain embodiments, the method further comprises: converting, using a transducer, the vibrations of the moveable member into an electrical signal; processing the electrical signal; and supplying the processed electrical signal to the one or more electrical speakers for generating the sound.

In certain embodiments, processing the electrical signal comprises improving a volume or quality of a voice encoded by the electrical signal.

Other aspects, features, and advantages will become apparent from the following Detailed Description when taken in conjunction with the accompanying drawings, which are a part of this disclosure and which illustrate, by way of example, principles of the various embodiments.

### BRIEF DESCRIPTION OF FIGURES

Example embodiments are apparent from the following description, which is given by way of example only, of at least one non-limiting embodiment, described in connection with the accompanying figures.

FIG. 1 illustrates a block diagram of an example voice generation system.

FIG. 2 illustrates a block diagram of another example voice generation system.

FIG. 3 illustrates a block diagram of another example voice generation system.

FIG. 4 illustrates a front perspective view of the voice generation system of FIG. 3 worn by a user.

FIG. 5 illustrates a side perspective view of the voice generation system of FIG. 3 worn by a user.

FIG. 6 illustrates a perspective view of a housing and a tube of the voice generation system of FIG. 3.

FIG. 7 illustrates a perspective view of a housing of the voice generation system of FIG. 3.

FIG. 8 illustrates an exploded view of the voice generation system of FIG. 3.

FIG. 9 illustrates an example moveable member of the voice generation system of FIG. 3.

FIG. 10 illustrates an example adhesive to attach the pressure sensing module of the voice generation system of FIG. 3 to the neck of a user.

FIG. 11 illustrates an example pressure sensing module of the voice generation system of FIG. 3 when attached to the neck of a user.

FIG. 12 illustrates a block diagram of another example voice generation system.

FIG. 13 illustrates the voice generation system of FIG. 12 in which a housing is supported by a neck harness.

FIG. 14 illustrates a block diagram of another example voice generation system.

FIG. 15 illustrates a block diagram of a mouth plate and a pressure/airflow sensing module of the voice generation system of FIG. 14 in wireless communication.

FIG. 16 illustrates an example method of generating a voice.

FIG. 17 illustrates an example of sound interference generation in the voice generation system.

FIG. 18 illustrates a block diagram of sound interference removal method in the voice generation system.

### DETAILED DESCRIPTION

The following modes, given by way of example only, are described in order to provide a more precise understanding of the subject matter of an embodiment or embodiments. In the figures, incorporated to illustrate features of an example embodiment, like reference numerals are used to identify like parts throughout the figures.

This invention describes an electronic respiratory driven voice generation system which generates a voice component and/or an airflow component for a person. The system acts as an artificial voice (and/or an airflow) source for the person to replace or augment the voice generation function of the vocal folds. The system may therefore be termed a “pneumatic bionic voice” source.

A “respiratory driven” voice generation system generates voice in response to variations of respiratory pressure or airflow. The system may provide access to these variations by monitoring the air pressure or airflow in front of the stoma of a larynx amputee person and/or inside their mouth.

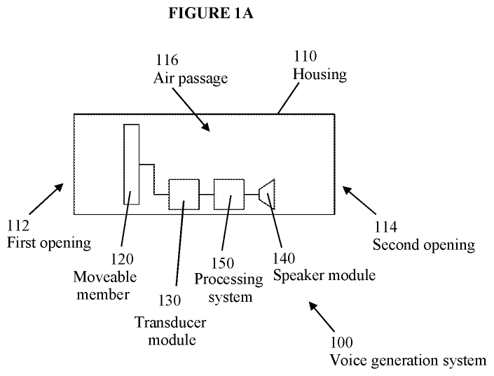

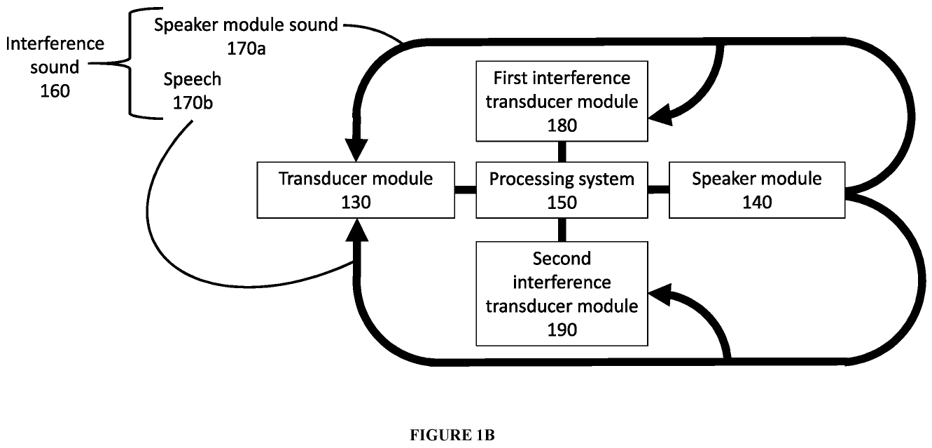

General embodiments of the voice generation system will now be described with reference to FIGS. 1A and 1B.

Referring to FIG. 1A, there is illustrated a voice generation system 100. Voice generation system 100 comprises a housing 110 comprising a first opening 112 and a second opening 114. Housing 110 defines an air passage, air channel, or air duct 116 between first opening 112 and second opening 114.

Voice generation system 100 further comprises a moveable member 120 located within housing 110 and configured to vibrate in response to air flowing in air passage 116 and/or in response to variations in air pressure at first opening 112 and second opening 114, or in air passage 116. In some examples, the moveable member 120 is located within air passage 116. In some examples, moveable member 120 extends transversely across air passage 116, in a direction orthogonal to the flow of air in air passage 116.

In some examples, moveable member 120 comprises a membrane. In some examples, moveable member 120 comprises a physical structure configured to vibrate in response to the air flowing in air passage 116. The physical structure of moveable member 120 may represent or conform to a mechanical model of human vocal folds with different levels of complexity.

Voice generation system 100 further comprises a transducer module 130. Transducer module may be located within housing 110. Transducer module 130 is configured to convert vibrations of moveable member 120 into an electrical signal. In some examples, transducer module 130 is physically coupled to moveable member 120. In other examples, transducer module 130 is operatively coupled to moveable member 120. In some examples, transducer module 130 comprises one or more microphones or a microphone array. In some examples, transducer module 130 comprises one or more piezoelectric transducers or magnetic pickup transducers, which have the advantage of reducing or avoiding audio interference from external sound sources. In some embodiments, transducer module 130 may comprise a sensor. This may be a pressure, sound, or vibration detection sensor. In some embodiments, transducer module 130 may comprise an accelerometer. In this specification, the term “transducer module” (including an interference transducer module) can include any of the examples mentioned. It may also include a voice detection sensor, sound sensor, vibration sensor, or the like.

Voice generation system 100 further comprises a speaker module 140. In some examples, speaker module is located within housing 110. In other examples, speaker module is not located within housing 110. Speaker module 140 is configured to convert the electrical signal generated by transducer module 130 into sound and to output the sound into the oral cavity of a user. In some examples, speaker module 140 comprises one or more loudspeakers or a loudspeaker array. In some examples, the loudspeaker's frequency response is flat (e.g. having less than 3 dB fluctuations) in the frequency range of human voice source (e.g. between about 50 Hz and about 1000 Hz).

In some examples, transducer module 130 and speaker module 140 are in communication, such as wired or wireless communication, to allow speaker module 140 to access the electrical signal produced by transducer module 130. In other examples, each of transducer module 130 and speaker module 140 is in communication with one or more processing systems (such as voice processing system 150 described below) which obtain the electrical signal from transducer module 130, optionally process or enhance the electrical signal (i.e. the voice signal), and then supply the voice as an electrical signal to speaker module 140 for conversion into sound.

In some embodiments, voice generation system 100 may be configured such that, during use, the air pressure at or near first opening 112 may be made to correspond to an air pressure at the neck of the user, such as for example the neck stoma of the user (or the air pressure in a respiratory airway, such as the trachea). The air pressure at or near the first opening 112 is considered to correspond to an air pressure at the neck (stoma) of the user if the first opening is in communication with the neck (stoma), or if air pressure at or near the first opening is generated artificially as an air pressure corresponding to (such as substantively the same, or substantively proportional for example) as the air pressure at the neck (stoma) of the user.

In some embodiments, it is desirable that the voice generation system 100 generates an output sound (from the speaker module for example) based on air pressure in the oral cavity or oral tract of the user. This can be done by:

making the air pressure at or near second opening 114 made to correspond to an air pressure in the oral cavity or the oral tract of the user; and/or

generating a voice based on monitored air pressure in the oral cavity or the oral tract, such as monitoring variations in air pressure in the oral cavity for example.

In some embodiments, the air pressure at or near second opening 114 may be made to correspond to an air pressure in the oral cavity or the oral tract of the user. The air pressure at or near the second opening 114 is considered to correspond to an air pressure in the oral cavity or oral tract of the user if the second opening is in communication with the oral cavity or tract, or if air pressure at or near the second opening is generated artificially as an air pressure corresponding to (such as substantively the same, or substantively proportional for example) the air pressure at the oral cavity or oral tract of the user.

It is advantageous for the air pressure at or near the second opening be made to correspond to the oral cavity or tract to improve quality of the sound output from the speaker module 140 and ultimately the quality of the speech output from the user. From experimentation, the inventors have found that speech output becomes monotonous if air pressure at or near the second opening 114 is not made to correspond to an air pressure in the oral cavity or oral tract of the user. The inventors have also found that an ability to control the transition the voice generation system between a voice mode and an unvoiced mode in real-time is desired. The inventors have found from experimentation that such control is compromised unless air pressure at or near the second opening is made to correspond to the oral cavity or tract as otherwise the moveable member will constantly produce vibrations as the person continues to exhale during speech. Similar advantages apply if more generally, the generated output sound (from the speaker module for example) is based on air pressure in the oral cavity or oral tract of the user.

Moreover, the advantages as discussed with respect to speech quality also applies if the voice generation system produces an output sound based on air pressure in the oral cavity of the user.

Moreover, the advantages as discussed with respect to speech quality is more apparent if in addition to generating an output sound (from the speaker module for example) based on air pressure in the oral cavity or oral tract of the user (such as the air pressure at or near the second opening being made to correspond to the oral cavity or tract), the air pressure at or near the first opening is made to correspond to the an air pressure at the neck of the user, such as the user's neck stoma for example. Therefore it is preferred that the voice generation system 100 is configured such that, during use, the air pressure at or near first opening 112 may be made to correspond to an air pressure at the neck of the user, such as for example the neck stoma (or the air pressure in a respiratory airway, such as the trachea), while the air pressure at or near second opening 114 may be made to correspond to an air pressure in the oral cavity or the oral tract of the user. Similar advantages apply if the voice generation system 100 is configured such that, during use, the air pressure at or near first opening 112 may be made to correspond to an air pressure at the neck of the user, such as for example the neck stoma (or the air pressure in a respiratory airway, such as the trachea), while the generated output sound (from the speaker module for example) is based on air pressure in the oral cavity or oral tract of the user.

As flow of air (may be referred to as “airflow” throughout the specification), is caused by a difference in air pressure, a situation where the airflow at or near the first end of the housing corresponds to an airflow at the neck (stoma) can be considered to be a situation where the air pressure at or near the first end of the housing corresponds to an air pressure at the neck (stoma). Similarly, a situation where the airflow at or near the second end of the housing corresponds to an airflow at the oral cavity or tract can be considered to be a situation where the air pressure at or near the second end of the housing corresponds to an air pressure in the oral cavity or tract.

A difference in the air pressures of the trachea (at the stoma) and the oral cavity thus may prompt a flow of air in passage 116 which causes moveable member 120 to vibrate. The vibrations of moveable member 120 are converted into one or more electrical signals, which are then transmitted to the speaker module for synthesising a voice in the oral cavity of the user. By shaping, filtering, or modifying the voice (or sound) produced by the speaker module using his or her lips, tongue, and vocal tract movements, the user is able to generate speech. Voice generation system 100 may therefore be termed a “pneumatic bionic voice” source.

The voice generation system and method described herein may generate an exceptionally high-quality voice for a person who has otherwise lost or damaged their larynx (vocal folds). The person uses the voice generation system as an artificial or augmented voice source to generate voice inside their oral cavity, which they use to modulate this voice, by moving their mouth and lips muscles to speak naturally. In doing so the voice generation system and method may provide an automated, real-time control of the voice onset and offset as the person uses the voice generation system to speak. The lack of onset/offset control results in generating voice in unvoiced phonemes or vice versa and negatively affects the intelligibility of the resulting speech.

In some examples, the onset or offset of the voice generated by the speaker module 140 is controlled using the variations of the air pressure of the mouth and stoma (such as, for example, that described by Ahmadi Farzaneh Ahmadi et al. “A pneumatic Bionic Voice prosthesis—Pre-clinical trials of controlling the voice onset and offset.” PloS one 13.2 (2018): e0192257). Using variations of respiration to control the voice onset and offset may help to avoid unwanted audio feedback between speaker module 140 and transducer module 130 and to reduce or avoid audio interference from external sound sources. This is described in more detail later.

Voice generation system 100 does not require surgical installation and is not invasive. Moreover, since voice generation system 100 is operable by the difference in air pressures at the stoma and oral cavity, the user becomes able to speak without the need for manual intervention.

In some examples, the air pressure/airflow at or near first opening 112 is set or generated naturally, for example, by arranging housing 110 such that first opening 112 is in connection or in communication with a neck stoma of the user, which is an opening in the front neck of the user in communication with the trachea so that the user inhales and exhales air through the stoma. In other examples, the air pressure at or near first opening 112 is set or generated artificially, for example, by providing housing 110 with an air pump that generates a pressure corresponding to the air pressure at the neck stoma. In some examples, a pressure sensor senses the air pressure or airflow at the neck stoma and the air pump is operated based on the sensed air pressure/airflow.

In some examples, the air pressure at or near second opening 114 is set or generated naturally, for example, by arranging housing 110 such that second opening 114 in connection or in communication with the oral cavity of the user. In other examples, the air pressure at or near second opening 114 is set or generated artificially, for example, by providing housing 110 with an air pump that generates a pressure corresponding to the air pressure in the oral cavity or in the vocal tract. In some examples, a pressure sensor senses the air pressure in the oral cavity and the air pump is operated based on the sensed air pressure.

An air micropump or any other airflow source may be used to generate a specified air pressure and/or airflow. In some examples, the airflow source comprises an array of micro-blowers or air nozzles. In some examples, the air pump or airflow source is able to generate an airflow rate of the respiratory drive of human voice, which is between about 5 litres per minute and about 10 litres per minute, or any other volume flow rate.

In addition to providing a sound (or voice), voice generation system 100 may further provide an airflow inside the oral cavity of the user in order to facilitate generating unvoiced phonemes (such as fricatives including, for example, /s/ or /f) in speech. In some examples, the airflow provided inside the oral cavity mirrors, copies, mimics, or reflects the airflow that travels through air passage 116 due to the pressure difference between first opening 112 and second opening 114. In some examples, the airflow provided inside the oral cavity is the same airflow that travels through air passage 116 due to the pressure difference between first opening 112 and second opening 114. In some examples, housing 110 is configured such that air passage 116 communicates with the oral cavity of the user to supply the airflow into the oral cavity.

In other examples, the airflow supplied into the oral cavity of the user is different and separate from the airflow that travels through air passage 116. In some examples, voice generation system 100 further comprises an air supply (such as an air pump) in communication with the oral cavity of the user and configured to generate a second airflow (other than the airflow through air passage 116) into the oral cavity.

Voice generation system 100 may further comprise a processing system 150. The processing system 150 may be configured to process the electrical signal generated by the transducer module. The processing system 150 may be configured to process the electrical signal generated by transducer module 130 to synthesize a higher quality or amplitude voice. Processing system 150 may be provided as part of transducer module 130, speaker module 140, or it may be distinct and separate from these two modules. In this specification, processing system 150, as well as the more general term “processing system”, may refer to one or more processors that may operate in tandem or independently. That is, various functions, or portions thereof that are performed by the processing system may be performed in a common processor, or across a plurality of processors.

In some examples, processing system 150 may run or execute a voice enhancement software to improve the quality of voice generated by voice generation system 100. Processing system 150 may be configured to enhance the voice through amplification, spectral enhancement, and/or pitch shifting into more advanced statistical voice conversion modules based on artificial intelligence (AI). The AI algorithms may be trained to improve the quality of the voice or even mimic the natural voice of the user. In some examples, the AI module is a statistical voice conversion module (such as, for example, that described by Toda, Tomoki, Alan W. Black, and Keiichi Tokuda, “Voice conversion based on maximum-likelihood estimation of spectral parameter trajectory”, IEEE Transactions on Audio, Speech, and Language Processing 15.8 (2007) 2222-2235), configured to convert the voice generated by moveable member 120 and picked up by the transducer module 130 in voice generation system 100 to be more natural-sounding. The AI algorithm may break the voice generated by transducer module 130 into three sets of parameters (the pitch (f0), spectral information and the aperiodicity). The algorithm may then convert these parameters to a more natural-sounding voice using trained AI statistical engines. Next, the algorithm may combine the estimated parameters (the pitch (f0), spectral information and the aperiodicity) with a vocoder to synthesize the enhanced, more natural-sounding voice. The algorithms may be trained with the natural voice of the user prior to the user undergoing a laryngectomy so that the algorithm can learn to convert the voice generated by the moveable member 120 to the user's natural voice.

The AI voice conversion module may be trained using mechanical or electromechanical pneumatic voice sources with more complicated shapes of moveable member 120 used by laryngectomy patients. By using more complicated moveable members such as mechanical models of vocal folds, including silicone membranes resembling the physical attributes of vocal folds, the voice generated by voice generation system 100 may sound closer to natural voice. However, driving more complicated pneumatic mechanical models may require more respiration effort from the patients, making it difficult for certain patients to drive the source. As such, sample wearable pneumatic complicated mechanical vocal fold models may be built, for example, with silicone models of vocal folds, and may be used by specific capable patients to generate voice in speech. A statistical voice conversion AI software may be trained to convert the voice generated by voice generation system 100 with a simple membrane as moveable member 120 (or its underlying respiration) into the voice that may be generated using these more complicated and more natural-sounding vocal fold models (membranes). The trained AI module may convert the simple (easy to drive) membrane's voice (or its underlying respiration) into a more natural sounding voice of complicated membranes of choice.

In some examples, there is provided a respiratory-driven electro-mechanic voice generation system. The voice generation system comprises a housing comprising a first opening and a second opening. The housing defines an air passage between the first opening and the second opening. The voice generation system further comprises a moveable member located within the housing and configured to vibrate in response to air flowing in the air passage. The voice generation system further comprises a transducer module configured to convert vibrations of the moveable member into an electrical signal. The voice generation system further comprises a speaker module configured to convert the electrical signal into sound and to output the sound into the oral cavity of a user.

In some examples, the voice generation system is respiratory-driven, and it generates voice in response to variations of respiratory pressure (and/or airflow) at the neck stoma and inside the oral cavity. In some examples, the voice generation system further comprises a voice enhancement module (such as processing system 150) between the transducer and the speaker modules. In some examples, the voice enhancement module is a hardware or software module that improves the quality or loudness of the sound.

In some examples, the voice generation system also generates or receives an air flow component in addition to the voice and outputs the voice and flow components into the oral cavity of a user. In some examples, the housing is configured to attach to the neck of the user such that the first opening 112 is in connection with the neck stoma.

In some examples, the voice generation system further comprises a pressure/airflow sensing module configured to attach to the neck of the user and to sense an air pressure or airflow at the neck stoma of the user. In some examples, the voice generation system further comprises a pressure/airflow sensing module configured to sense an air pressure or airflow inside the mouth. In some examples the pressure/flow sensing module samples the respiration signal at 1 kHz.

In some embodiments, it is desirable to reduce (sound) interference (such as acoustic feedback or echo for example) received at the transducer module. Such interference can be due to undesired feedback from the sound output from the speaker module being picked up by the transducer module. In addition, such interference can also be caused by undesired feedback from speech signal being picked up by the transducer module, whereby the sound output from the speaker module is modulated by movement of the user's mouth or lips. Such interference is especially problematic if it dilutes the electrical signal of the transducer module converted from the vibrations of the moveable member and becomes amplified as this results in undesired positive feedback. In these situations, the processing system 150 can be configured to reduce sound interference received at the transducer module 130. Having the processing system 150 can be configured to reduce sound interference received at the transducer module is advantageous as it ultimately improves quality of sound output from the speaker module 140.

In this specification the reduction of interference sound can be considered to at least include, but not be restricted to, the minimisation, cancellation, suppression, avoidance, or elimination of interference sound.

In this specification, “sound interference”, interchangeable with “interference sound” is meant to include but is not limited to acoustic feedback, echo, ambient noise, undesired feedback sound originating from the speaker module 140 or speech signal that is generated when the sound outputted from the speaker module into the oral cavity of the user excited the user's vocal tract.

Turning to FIG. 1B by way of example, it is desirable to reduce the interference sound 160 (interchangeable with “sound interference” in this specification). Interference sound 160 can include undesired feedback sound originating from the speaker module 140, including speaker module sound 170a and/or speech 170b. For context, speech 170b is the sound that is generated when the sound outputted from the speaker module into the oral cavity of the user excites the user's vocal (oral) tract. However, interference sound 160 can include other forms of undesired interference sounds from other sources. Interference sound 160 can also include (acoustic) echoes or acoustic feedback.

In some embodiments, the processing system 150 is configured to reduce sound interference 160 based on at least one electrical signal received from the transducer module 130 configured to convert vibrations of the moveable member, and/or at least one interference transducer module for receiving sound interference, such as interference transducer module 180 and/or interference transducer module 190 for example.

In some embodiments, processing system 150 can receive an electrical input signal from the transducer module 130 to reduce sound interference. In such embodiments, the interference sound 160 that is picked up by the transducer module 130 is used by the processing system 150 to reduce interference sound.

Additionally, or alternatively, the processing system 150 can receive an electrical input signal from at least one interference transducer module (that is different to transducer module 130) to identify and reduce sound interference. In such embodiments, the interference sound 160 that is picked up by the at least one interference transducer module is used by the processing system 150 to reduce interference sound. For example, speaker module sound 170a that is picked up by a first interference transducer module 180 can be used by the processing system 150 to reduce interference sound. In another example, the processing system 150 can be used to reduce interference sound when a first interference transducer module 180 picks up the speaker module sound 170a, and a second interference transducer module 190 picks up speech 170b. Additional interference transducer modules can be used if desired.

In some examples, the interference transducer module (including first and/or second transducer module 180,190) comprises one or more microphones or a microphone array. In some examples, the interference transducer module (including first and/or second transducer module 180,190) comprises one or more of: piezoelectric transducers, magnetic pickup transducers, accelerometers, or any other voice, sound, or vibration sensor, which have the advantage of identifying or reducing audio interference from external sound sources.

In some embodiments, the processing system 150 is configured to reduce sound interference by performing feedforward suppression (interchangeable with “forward suppression”). In such embodiments, feedforward suppression may comprise one or more decorrelation operations, such as for example: gain reduction, frequency selective gain reduction, frequency selective noise reduction, notch filter, phase modulation and frequency shifting. In some embodiments, the processing system 150 is configured to reduce sound interference by performing adaptive feedback cancellation. In such embodiments, adaptive feedback cancellation may comprise adaptive filtering. Optionally, adaptive filtering is done with an FIR filter. Optionally, adaptive filtering is done using a (real-time) normalized least-mean-square (NLMS) algorithm. In some embodiments, the processing system 150 is configured to reduce sound interference by performing residual feedback suppression.

In some embodiments the processing system may include an AI algorithm that is trained to remove the sound interference from the transducer signal. This can be done with or without additional interference transducer.

In some embodiments, there may be provided a physical connection (e.g. a pneumatic connection) between the moveable member, the user's mouth, and the user's stoma (such that they are all in connection with each other). This may lead to saliva leaking from the mouth to the stoma (and possibly penetrating the moveable member such as the membrane tube).

To avoid that in such embodiments the design may include a moisture sensor which detects saliva leakage or water presence close to the stoma. In case such saliva presence is detected the voice generation system stops voice generation with an indicator light flashing to warn the user of saliva presence to clean the voice generation system.

In some embodiments, the voice generation system is configured to be used hands free.

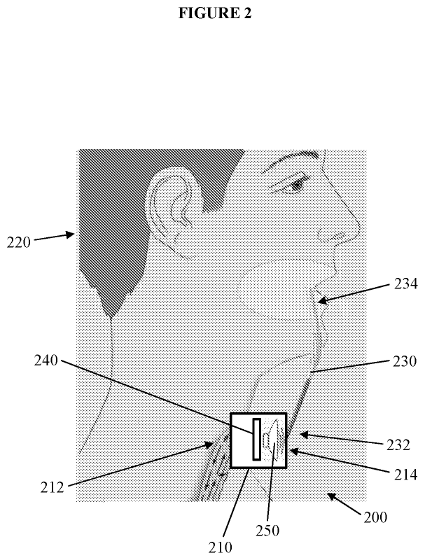

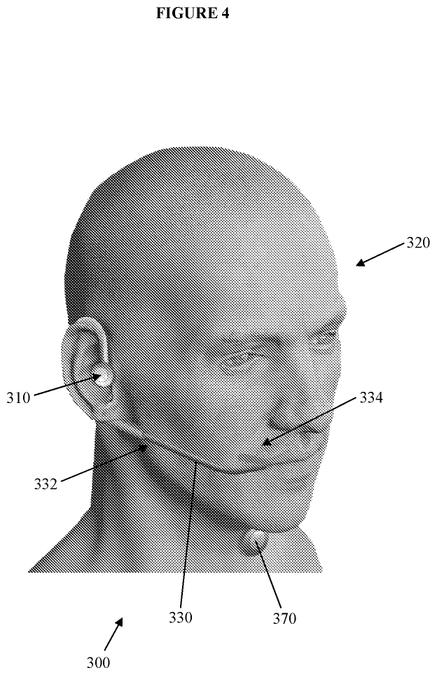

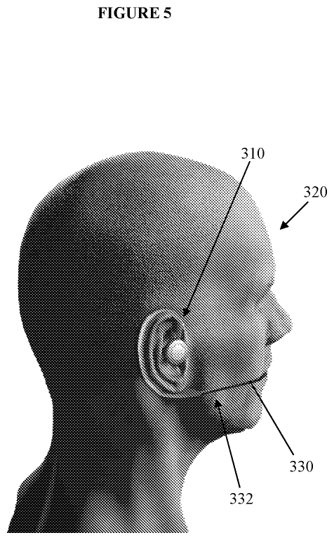

Up until this point, the detailed description has described general embodiments of the voice generation system with respect FIG. 1A and 1B. Exemplary voice generation system embodiments will now be described with reference to FIGS. 2-18.

Referring to FIG. 2, there is illustrated another example voice generation system 200.

Voice generation system 200 comprises a housing 210 comprising a first opening 212 and a second opening 214. Housing 210 defines an air passage between the first opening 212 and the second opening 214. The first opening 212 is configured to be in communication with a neck stoma of a user 220. In some examples, housing 210 is configured to attach to the neck of user 220 such that the first opening 212 is in connection with the neck stoma. The second opening 214 is configured to be in communication with the oral cavity of user 220.

Therefore, housing 210 is configured to attach to the neck of user 220 such that the first opening 212 is in connection or in contact with the neck stoma.

Voice generation system 200 further comprises a tube 230 comprising a first open end 232 connected to housing 210 around the second opening, and a second open end 234 of the tube configured to be inserted into the oral cavity of user 220. In some examples, tube 230 is a disposable tube.

Tube 230 is hollow and defines a second air passage (for both a voice and an airflow) between first and second ends 232 and 234. In some examples, tube 230 defines a voice passage and an airflow passage that are separate from each other. The air passage of tube 230 is in communication with the air passage of housing 210. Therefore, housing 210 and tube 230 together define an air passage between the first opening 212 of housing 210 and the second end 234 of tube 230. During use, air ejected from the neck stoma during a respiratory exhalation travels along the air passages defined by housing 210 and tube 230 and is outputted into the oral cavity of user 220.

Tube 230 may comprise one or more filters or bends (e.g. a membrane air filter or U-shape) to prevent or restrict food residues, saliva, or other fluids in the oral cavity entering second end 234 from reaching the first opening 212 of housing 210 and, consequently, the trachea of user 220.

The air pressure at the first opening 212 may correspond to an air pressure in the neck stoma, while an air pressure at the second opening may correspond to an air pressure in the oral cavity of user 220.

Housing 210 and tube 230 together define an air passage extending between the neck stoma and the oral cavity of user 220. In this way, respiration airflow enters the first opening 212 of housing 210 and is configured to propagate along the air passage of housing 210, along the air passage of tube 240, and be outputted into the mouth or oral cavity of user 220.

Voice generation system 200 further comprises a moveable member 240 located within the housing and configured to vibrate in response to respiration air flowing in the air passage of housing 210.

Voice generation system 200 further comprises a transducer module configured to convert vibrations of moveable member 240 into an electrical signal.

In some examples, voice generation system 200 further comprises a processing system including voice enhancement software/hardware to improve the quality or loudness of the voice generated by voice generation system 200. Artificial intelligence (AI) algorithms may be used in the processing system to learn to improve the quality of the voice or even mimic the natural voice of the user.

Voice generation system 200 further comprises a speaker module 250 located within housing 210. Speaker module 250 is configured to convert the electrical signal representing the voice (received from the transducer module or from the voice enhancement module) into sound and to output the sound into the oral cavity of user 220. Speaker module 250 is configured to output the sound through the second opening of housing 210. In some examples, the speaker module is orientated to output sound into tube 240 to project the sound into the oral cavity of user 230.

Therefore, in some examples, voice generation system 200 couples a the naturally-generated air pressure in the neck stoma and a naturally-generated air pressure in the oral cavity of user 220 to moveable member 240. In some examples, voice generation system 200 couples a natural airflow from respiration to moveable member 240. The resulting airflow, or air pressure gradient, experienced by moveable member 240 causes it to vibrate.

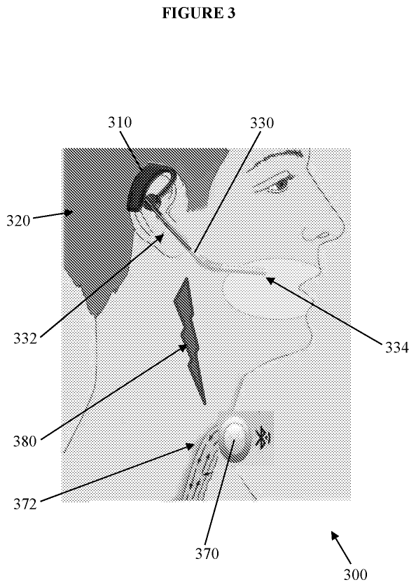

Referring to FIGS. 3 to 8, there is illustrated another example voice generation system 300.

Voice generation system 300 comprises a housing 310 comprising a first opening and a second opening. Housing 310 defines an air passage between the first opening and the second opening. The first opening is an airhole or an air intake configured to admit air into housing 310 for feeding an air pump located within housing 310, while the second opening is configured to be in communication with the oral cavity of user 320.

Housing 310 may be configured to be secured to an auricle, or a projecting outer portion of the ear, of user 320, and is placed between the auricle and the head of user 320. In some examples, housing 310 has an auricular or spiral shape to facilitate securing housing 310 to an auricle of a person. In other examples, housing 310 is a handheld housing.

Voice generation system 300 further comprises a tube 330 comprising a first open end 332 connected to housing 310 around the second opening, and a second open end 334 configured to be inserted into the oral cavity of user 320. When housing 310 is secured to the auricle, tube 330 is configured to bend around an exterior side of a cheek of user 320 and reach into the mouth of user 320.

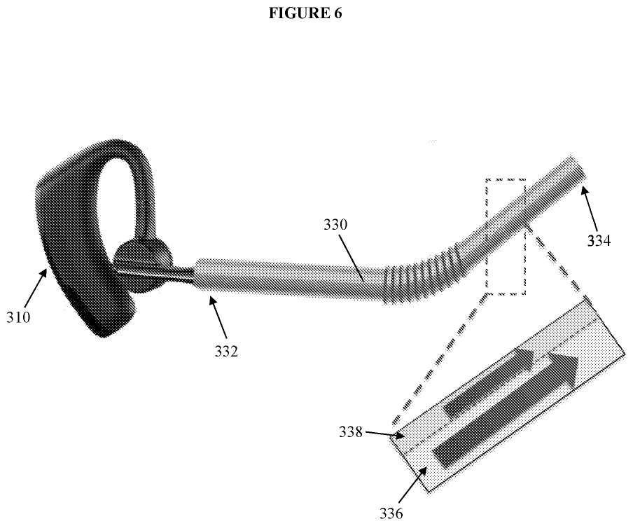

Tube 330 is hollow and defines a second air passage (for both a voice and an airflow) between first and second ends 332 and 334. The air passage of tube 330 is in communication with the air passage of housing 310. Therefore, housing 310 and tube 330 together define an air passage between the first opening of housing 310 and the second end (or mouthpiece end) of tube 330. In some examples, as illustrated in FIG. 6, tube 330 defines a voice passage 336, configured to transport a voice sound, and an airflow passage 338, configured to transport a flow of air. Voice passage 336 and airflow passage 338 may be separate from each other.

Voice generation system 300 further comprises an air pump 350 located within housing 310. Air pump 350 is configured to generate an airflow that moves along the air passage from the first opening to the second opening.

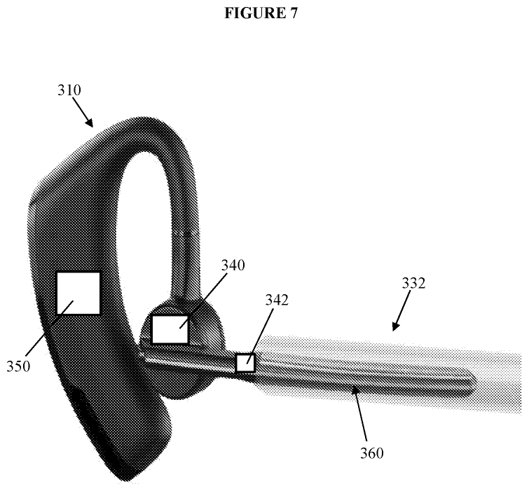

Voice generation system 300 further comprises a moveable member 340, illustrated in FIG. 7, located within the housing and configured to vibrate in response to air flowing in the air passage of housing 310, the airflow in the air passage being generated by air pump 350.

Voice generation system 300 further comprises a transducer module 342 configured to convert vibrations of moveable member 340 into an electrical signal. As illustrated in FIG. 7, transducer module 342 may be located within housing 310, and be operatively coupled to moveable member 340.

Voice generation system 300 may further comprise, a processing system including a voice enhancement software/hardware module to improve the quality or loudness of the voice generated by moveable member 340 and picked up via transducer module 342. The processing system may use Artificial intelligence (AI) to learn to improve the quality of the voice or even mimic the natural voice of the user.

Voice generation system 300 further comprises a speaker module 360 located within housing 310. Speaker module 360 is configured to convert the electrical signal representing the voice (received from transducer module 342 or the voice enhancement module) into sound and to output the sound into the oral cavity of user 320. Speaker module 360 is configured to output the sound through the second opening of housing 310. As illustrated in FIG. 7, speaker module 360 may comprise a speaker array distributed along an elongate tubular portion of housing 310 in proximity of the second opening so as to better direct the sound into tube 330.

Voice generation system 300 further comprises a pressure sensing module 370 configured to attach to the neck of user 320. Pressure sensing module 370 is configured to sense an air pressure (and/or air flow) at a neck stoma of user 320. Pressure sensing module 370 is fixed over a stoma or opening 372 in the anterior neck of user 320.

Voice generation system 300 further comprises a controller located within housing 310. The controller is configured to control air pump 350 based on the sensed air pressure/air flow at the neck stoma in real-time. The controller is operatively coupled to air pump 350 in order to actuate or operate air pump 350. In some examples, the controller is located within housing 310. In other examples, the controller forms part of a processing system.

The controller may be configured to receive or obtain a measurement, reading, or indication of an air pressure from pressure sensing module 370. In some examples the pressure sensing module samples the respiration signal at 1 kHz and sends it to the controller. In some examples, the controller is in wireless communication with pressure sensing module 370, such as through a Bluetooth wireless link 380 and a connectionless UDP protocol. In some examples, pressure sensing module 370 comprises a wireless transmitter, such as a Bluetooth transmitter, and the controller comprises a wireless receiver, such as a Bluetooth receiver, for enabling wireless communication between the controller and pressure sensing module 370.

The controller may control air pump 350 such that a pump output is proportional, or otherwise dependent on, or a function of, the magnitude of the air pressure sensed by pressure sensing module 370. In some examples, air pump 350 is configured to generate an air pressure and/or an airflow in the air passage corresponding to the air pressure or airflow at the neck stoma measured by pressure sensing module 370. In some examples, the controller has less than 5 milliseconds of delay to follow of the air pressure/flow in the neck stoma to maintain a real-time performance.

The controller may control air pump 350 to generate an airflow with different characteristics (e.g. airflow volume, airflow rate) based on the sensed breathing air pressure. Moreover, the characteristics of the airflow may affect characteristics of the sound generated by moveable member 340 as it vibrates (e.g. pitch, frequency spectrum, or volume). In this way, the sound generated by moveable member 340 is dependent on the breathing air pressure of user 320, as well as the physical shape and material used in moveable member 340.

Therefore, voice generation system 300 monitors the respiratory airflow of user 320 at the neck stoma and recreates it (in terms of air pressure and/or airflow) by sending wireless commands to air pump 350 placed inside housing 310. Air pump 350 may therefore generate an air pressure and/or an airflow that corresponds, is similar, or is otherwise related to the breathing air pressure and/or airflow of user 320.

In this way, the air flow in the air passage in housing 310 may correspond to an air pressure generated by the air pump 350, which in some examples represents the air pressure of the neck stoma, and the air pressure at the second opening which may correspond to an air pressure in the oral cavity of user 320. Hence the moveable member is affected on one side by the air pressure at the neck stoma generated artificially using air pump 350 and on the other side by the air pressure in the oral cavity that is generated naturally by user 320. The resulting airflow, or air pressure gradient, experienced by moveable member 340 causes it to vibrate and generate a voice.

Moreover, the airflow generated by air pump 350 is configured to propagate along the air passage of housing 310, along the air passage of tube 330, and be outputted into the mouth or oral cavity of user 320 to provide the air flow to generate consonants in speech.

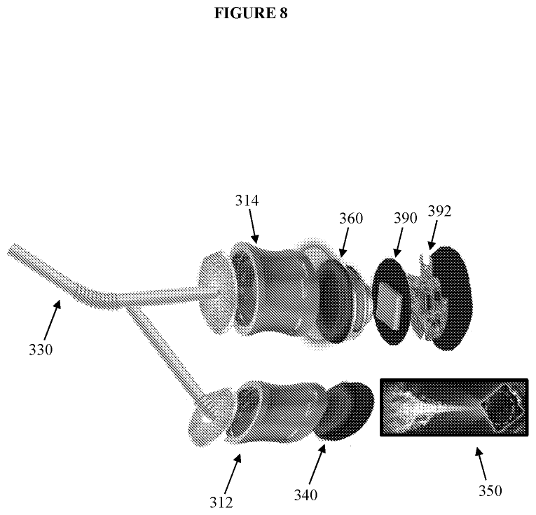

In some examples, as illustrated in FIG. 8, the airflow and the sound are generated in two different sections or compartments of housing 310. Housing 310 comprises a first compartment 312 for generating the airflow and a second compartment for synthesising the voice. Compartment 312 contains moveable member 340, the transducer module, and air pump 350, while compartment 314 contains speaker module 360, as well as electronics for the operation of voice generation system 300, such as a battery 390 and a voice enhancement and/or conversion unit 392 (including hardware and software) comprising a processing system for processing the electrical signal adjusting the sound quality produced by speaker module 360.

Compartments 312 and 314 may be coupled or linked so as to be in communication with tube 330. In this way, the airflow and the sound can be combined and such that both travel into the oral cavity of user 320 along the same air passage.

Advantageously, voice generation system 300 does not necessitate a physical channel between the mouth and breathing airway (stoma) of user 320.

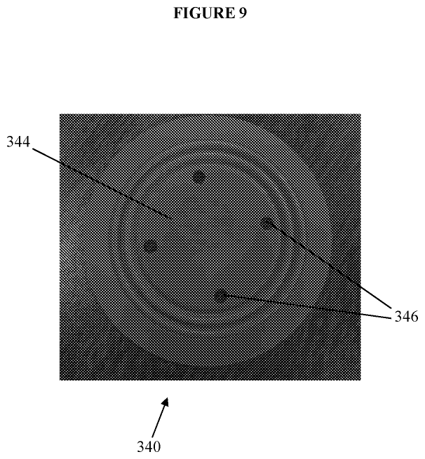

Referring to FIG. 9, there is illustrated an example moveable member 340 suitable for use with voice generation system 300 or with any other voice generation system. Moveable member 340 comprises a single membrane 344. Membrane 344 is circular in shape. A plurality of apertures 346 are disposed on an outer rim of membrane 344 to allow membrane 344 to be fixed to an interior volume the housing of a voice generation system. In other examples, membrane 344 may have any other shape or may represent a simple or complicated mechanical model of the vocal folds such as synthetic multi-layer silicone model of vocal folds (such as, for example, that described in Murray, Preston R., and Scott L. Thomson, “Synthetic, multi-layer, self-oscillating vocal fold model fabrication”, JoVE (Journal of Visualized Experiments) 58 (2011): e3498). Membrane 344 may be made of silicone, natural rubber, or any other flexible material, and may comprise a single layer or multiple layers (e.g. filled with gel-type material). Moveable member 340 may include one or more vibrating elements and may have a non-uniform 3D pattern to generate irregular vibrations for the resulting voice to sound less monotonous and more natural.

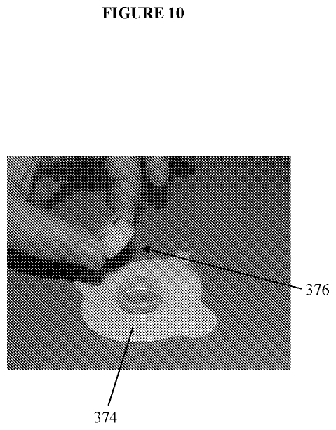

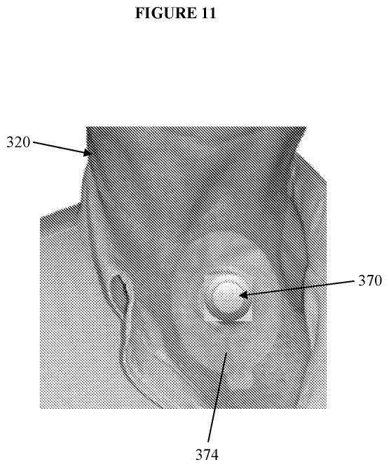

Referring to FIGS. 10 and 11, there is illustrated an adhesive to attach an example pressure sensing module 370 to the neck of a user. Pressure sensing module 370 may be suitable for use with voice generation system 300 or with any other voice generation system. Pressure sensing module 370 is configured to be mounted or coupled to an adhesive strip 374 over a hole in strip 374. Strip 374 comprises an adhesive surface configured to adhere to the skin of user 320 for attaching pressure sensing module 370 to the neck or chest of user 320, over a neck stoma. The strip 374 may hold a heat and moisture exchangers (HME) cap 376 over the stoma of a larynx amputee person to protect their airway from external pollution. Pressure sensing module 370 may be placed inside HME cap 376.

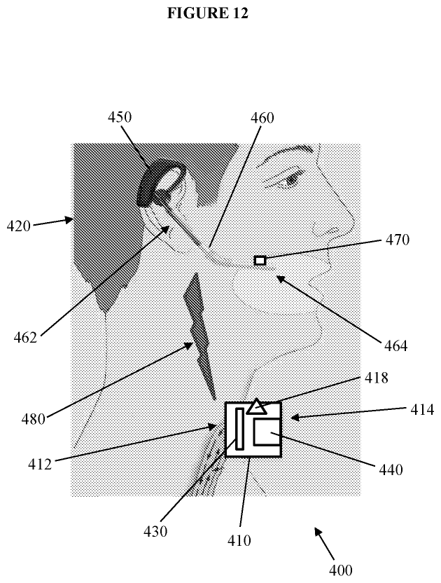

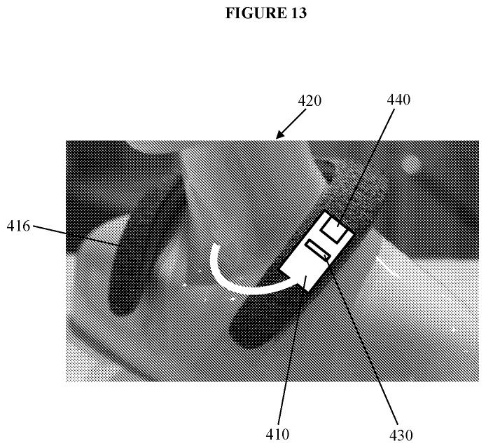

Referring to FIGS. 12 and 13, there is illustrated another example voice generation system 400.

Voice generation system 400 comprises a first housing 410 comprising a first opening 412 and a second opening 414. Housing 410 defines an air passage between the first opening 412 and the second opening 414. The first opening 412 is configured to be in communication with a neck stoma of user 420, while the second opening 414 may be in communication with an external environment outside of housing 410 and user 420 or with a sound muffler (as explained below). Therefore, the air flow to first opening 412 corresponds to the air flow of the neck stoma of user 420 due to respiration.

In some examples, as illustrated in FIG. 12, housing 410 is configured to attach directly to the front neck of a user 420 such that the first opening is in connection with, or contiguous to, the neck stoma. In other examples, as illustrated in FIG. 13, housing 410 is fixed to, or integrated into, a neck harness 416, comprising an arched or U-shaped frame configured to rest on the shoulders and around the rear neck of user 420.

Voice generation system 400 further comprises a moveable member 430 located within housing 410 between first opening 412 and second opening 414, and configured to vibrate in response to air flowing in the air passage of housing 410.

Voice generation system 400 further comprises a transducer module configured to convert vibrations of moveable member 430 into an electrical signal. In some examples, transducer module is located within housing 410.

In some embodiments of voice generation system 400 where the second opening 414 may be connected to open air, the moveable member sound becomes monotonous since air pressure at or near the second opening 414 is not connected to the oral cavity or oral tract. To resolve this issue, the oral pressure airflow can be monitored to improve and adjust the quality of the voice signal. Hence, the voice generation system 400 is still strongly influenced by the variations of pressure at the mouth to maintain a non-monotonous voice. That is, in these embodiments, the voice generation system produces a sound output that is based on monitored air pressure.

Voice generation system 400 further comprises a regulator, or airflow control element, 440 located within housing 410 and configured to control a flow of air in the air passage.

In some examples, regulator 440 is a first air pump that is configured to generate an air pressure and/or an airflow in the air passage at or near second opening 414. The air pressure generated by the first air pump may correspond to an air pressure in the oral cavity or the oral tract of user 420. The difference between the stoma air pressure at first opening 412 and the pressure generated by the first air pump at second opening 414 produces an airflow that moves along the air passage from the first opening 412 to the second opening 414 and vibrates moveable member 430.

In other examples, regulator 440 is an air valve configured to control the flow of air through the second opening 414. The air valve may allow for the regulation of air flow into and out of the air passage through second opening 414. In some examples, the air valve is an electromechanically operated valve such as a miniature solenoid valve, which may provide a controlled gateway for air that modifies (e.g. opens and closes) the air passage in a linear fashion with low delay (e.g. milliseconds delay) and in a quiet mode with low audible noise (e.g. 20 dB). The air valve may increase or decrease the airflow by being actuated between an open state and a closed state. The air valve may also have one or more partially opened or partially closed states to provide continuous or fine control of fluid flow between the open state and the closed state. Thus, by operating the air valve, the pressure and/or airflow in the air passage in vicinity of second opening 414 can be controlled. The respiratory airflow and/or pressure produced by user 420 through the neck stoma when regulated by the air valve vibrates the moveable member 430. In other examples the regulator 440 is a suction. In other examples the regulator 440 is an actuator. The actuator may be a mini actuator or a micro actuator.

Voice generation system 400 further comprises a second housing 450 which may be configured to be secured to an auricle of user 420. Housing 450 is separate and distinct from housing 410. Housing 450 comprises a first opening which is an airhole or an air intake configured to admit air into housing 450 for feeding an air pump (i.e. a second air pump of voice generation system 400) contained therein, and a second opening in communication with the oral cavity of user 420. Housing 450 defines an air passage between the first opening and the second opening. In other examples, housing 450 comprises a single opening, the single opening being in communication with the oral cavity of user 420.

Voice generation system 400 further comprises a tube 460 comprising a first open end 462 connected to the second opening of housing 450, and a second open end 464 configured to be inserted into the oral cavity of user 420.

Voice generation system 400 further comprises an air pump located within housing 450. The air pump of housing 450 may be termed a second air pump to distinguish it from the first air pump of regulator 440. The second air pump is configured to generate an airflow into the oral cavity of user 420. In some examples, the airflow generated by the second air pump corresponds to or represents an air flow from the neck stoma, due to respiration, in housing 410.

Voice generation system 400 further comprises a flow sensing module 418 located within housing 410. In some examples, flow sensing module 418 is located within the air passage of housing 410. Flow sensing module 418 is configured to sense or measure the air flow (such as the air flow rate and/or volume) in the neck stoma or in the air passage of housing 410 from the neck stoma, due to respiration. In some examples, flow sensing module 418 comprises a differential pressure sensor, or two or more pressure sensors. In some examples, the measurement of the flow rate performed by flow sensing module 418 is used to operate the second air pump within housing 450 to generate an air flow corresponding to the air flow from the neck stoma.

Voice generation system 400 further comprises a speaker module located within housing 450. The speaker module is configured to convert the electrical signal generated by the transducer module into voice and to output the voice into the oral cavity of user 420. The speaker module is configured to output the voice through the second opening of housing 450. In some examples, speaker module 450 is configured to receive electrical signals generated by the transducer module (representing the voice) via a wired or wireless communication link between housing 410 and housing 450.

In some examples, it may be desirable to suppress sound generated by the vibration of moveable member 430 since this sound may interfere with or disturb the sound generated by the speaker module (representing the voice). Therefore, in some examples, voice generation system 400 further comprises a sound muffler or other noise reducing element to reduce or suppress unwanted background noise generated by the vibration of moveable member 430. The sound muffler may be provided within first housing 410, such as in the air passage, or may be connected to second opening 414.

Voice generation system 400 further comprises a pressure sensing module 470 configured to sense the air pressure of the oral cavity of user 420. In some examples, pressure sensing module 470 is configured to measure a pressure signal in the oral cavity. In some examples, pressure sensing module 470 is fixed to the second end of tube 460 so as to be located within the oral cavity of user 420 during use. In some examples, pressure sensing module 470 comprises an intra-oral pressure sensor.

Voice generation system 400 further comprises a controller located within housing 410. The controller is configured to control regulator 440 based on the air pressure in the oral cavity sensed by pressure sensing module 470. In some examples, the controller is in wireless communication with pressure sensing module 470, such as through a Bluetooth wireless link 480. In some examples, pressure sensing module 470 comprises a wireless transmitter, such as a Bluetooth transmitter, and the controller comprises a wireless receiver, such as a Bluetooth receiver, for enabling wireless communication between the controller and pressure sensing module 470.

Voice generation system 400 may further comprise a second controller located within second housing 450. The second controller is configured to control the second air pump based on the sensed air flow from the neck stoma measured by flow sensing module 418. In some examples, the second controller is in wireless communication with the flow sensing module 418, such as through a second Bluetooth wireless link. In some examples, flow sensing module 418 comprises a wireless transmitter, such as a Bluetooth transmitter, and the second controller comprises a wireless receiver, such as a Bluetooth receiver for enabling wireless communication between the second controller and flow sensing module 418.

In some examples, when regulator 440 is an air pump, the air pump is configured to generate an air pressure in the air passage corresponding to the air pressure in the oral cavity measured by pressure sensing module 470. In other examples, when regulator 440 is an air valve, it is configured to control the flow of air in the air passage based on the air pressure in the oral cavity measured by pressure sensing module 470 (the air pressure of the oral cavity may have an inverse relationship to or influence on the airflow of the valve such that when the air pressure in the oral cavity increases the flow of air decreases).

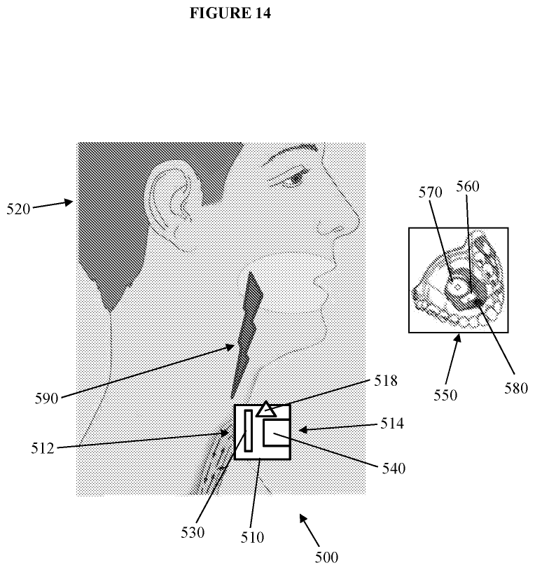

Referring to FIG. 14, there is illustrated another voice generation system 500.

Voice generation system 500 comprises a first housing 510 comprising a first opening 512 and a second opening 514. Housing 510 defines an air passage between the first opening 512 and the second opening 514. The first opening 512 is configured to be in communication with a neck stoma of user 520, while the second opening 514 may be in communication with an external environment outside of housing 510 and user 520 or with a sound muffler (as described above). Therefore, the air flow to first opening 512 corresponds to the air flow of the neck stoma of user 520.

In some examples, housing 510 is configured to attach directly to the front neck of a user 520 such that the first opening is in connection with, or contiguous to, the neck stoma. In other examples, housing 510 is fixed to, or integrated into, a neck harness, comprising an arched or U-shaped frame configured to rest on the shoulders and around the rear neck of user 520 (in an arrangement similar to that illustrated in FIG. 13).

Voice generation system 500 further comprises a moveable member 530 located within housing 510 between first opening 512 and second opening 514 and configured to vibrate in response to air flowing in the air passage of housing 510.

Voice generation system 500 further comprises a transducer module configured to convert vibrations of moveable member 530 into an electrical signal. In some examples, transducer module is located within housing 510.

In some embodiments of voice generation system 500 where the second opening 514 may be connected to open air, the moveable member sound becomes monotonous since air pressure at or near the second opening 514 is not connected to the oral cavity or oral tract. To resolve this issue, the oral pressure airflow can be monitored to improve and adjust the quality of the voice signal. Hence, the voice generation system 500 is still strongly influenced by the variations of pressure at the mouth to maintain a non-monotonous voice. That is, in these embodiments, the voice generation system produces a sound output that is based on monitored air pressure.

Voice generation system 500 further comprises a regulator, or airflow control element, 540 located within housing 510 and configured to control a flow of air in the air passage.

In some examples, regulator 540 is a first air pump that is configured to generate an air pressure and/or an airflow in the air passage at or near second opening 514. The air pressure generated by the first air pump may correspond to an air pressure in the oral cavity or the oral tract of user 520. The difference between the stoma air pressure at first opening 512 and the pressure generated by the first air pump at second opening 514 produces an airflow that moves along the air passage from first opening 512 to second opening 514 and vibrates the moveable member 530.

In other examples, regulator 540 is an air valve configured to control the flow of air through the second opening 514. The air valve may allow for the regulation of air flow into and out of the air passage through second opening 514. In some examples, the air valve is an electromechanically operated valve such as a miniature solenoid valve, which may provide a controlled gateway for air that modifies (e.g. opens and closes) the air passage in a linear fashion with low delay (e.g. milliseconds delay) and in a quiet mode with low audible noise (e.g. 20 dB). The air valve may increase or decrease the airflow by being actuated between an open state and a closed state. The air valve may also have one or more partially opened or partially closed states to provide continuous or fine control of fluid flow between the open state and the closed state. Thus, by operating the air valve, the pressure and/or airflow in the air passage in vicinity of second opening 514 can be controlled. The respiratory airflow and/or pressure produced by user 520 through the neck stoma and regulated by the air valve vibrates the moveable member 530. In other examples the regulator 540 is a suction. In other examples the regulator 540 is an actuator. The actuator may be a mini actuator or a micro actuator.

Voice generation system 500 further comprises a denture unit, mouth plate, or frame 550 which is configured to be secured to the oral cavity of user 520, for example, in the palate. Mouth plate 550 is separate and distinct from housing 510. Mouth plate 550 comprises a frame that is open to, or in communication with, the oral cavity of user 520.

Voice generation system 500 further comprises an air pump 560 connected to mouth plate 550. The air pump of mouth plate 550 may be termed a second air pump to distinguish it from the first air pump of regulator 540. In some examples, air pump 560 is a micro-air pump. Air pump 560 is configured to generate an airflow into the oral cavity of user 520. In some examples, the airflow generated by second air pump 560 corresponds to or represents an air flow from the neck stoma, due to respiration, measured in housing 510.

Voice generation system 500 further comprises a flow sensing module 518 located within housing 510. In some examples, flow sensing module 518 is located within the air passage of housing 510. Flow sensing module 518 is configured to sense or measure the air flow (such as the air flow rate and/or volume) in the neck stoma or in the air passage of housing 510 from the neck stoma, due to respiration. In some examples, flow sensing module 518 comprises a differential pressure sensor, or two or more pressure sensors. In some examples, the measurement of the flow rate performed by flow sensing module 518 is use to operate second air pump 560 to generate an air flow corresponding to the air flow from the neck stoma.

Voice generation system 500 further comprises a speaker module 570 fixed to mouth plate 550. Speaker module 570 is configured to convert the electrical signal generated by the transducer module into sound and to output the sound into the oral cavity of user 520. In some examples, speaker module 570 is configured to receive electrical signals generated by the transducer module (representing the voice) via a wired or wireless communication link between housing 510 and mouth plate 550.

Voice generation system 500 further comprises a pressure sensing module 580 fixed to mouth plate 550 (denture source). Pressure sensing module 580 is configured to sense an air pressure in the oral cavity of user 520. In some examples, pressure sensing module 580 is configured to sample a respiration signal in the oral cavity. In some examples, pressure sensing module 580 comprises an intra-oral pressure sensor.

Voice generation system 500 further comprises a controller located within housing 510. The controller is configured to control regulator 540 based on the sensed air pressure in the oral cavity. In some examples, the controller is in wireless communication with pressure sensing module 580, such as through a Bluetooth wireless link 590. In some examples, pressure sensing module 580 comprises a wireless transmitter, such as a Bluetooth transmitter, and the controller comprises a wireless receiver, such as a Bluetooth receiver, for enabling wireless communication between the controller and pressure sensing module 580.

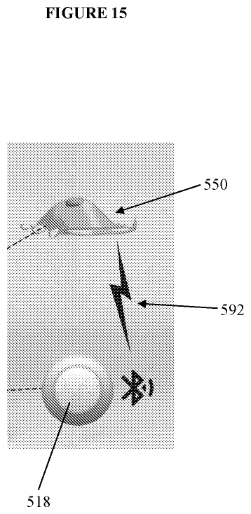

Voice generation system 500 may further comprise a second controller fixed to mouth plate 550. The second controller is configured to control second air pump 560 based on the sensed air flow from the neck stoma measured by flow sensing module 518. In some examples, the second controller is in wireless communication with the flow sensing module 518, such as through a second Bluetooth wireless link 592, as illustrated in FIG. 15. In some examples, flow sensing module 518 comprises a wireless transmitter, such as a Bluetooth transmitter, and the second controller comprises a wireless receiver, such as a Bluetooth receiver for enabling wireless communication between the second controller and flow sensing module 518.

In some examples, when regulator 540 is an air pump, the air pump is configured to generate an air pressure in the air passage corresponding to the pressure in the oral cavity measured by pressure sensing module 580. In other examples, when regulator 540 is an air valve, it is configured to control the flow of air in the air passage based on the air pressure in the oral cavity measured by pressure sensing module 580 (the air pressure of the oral cavity may have an inverse relationship to or influence on the airflow of the valve, such that when the air pressure in the oral cavity increases the flow of air decreases).

Voice generation systems 400 and 500 employ a “push-pull” mechanism to induce vibrations in the moveable member. That is, an airflow exiting the neck stoma during a respiration exhalation “pushes” on the moveable member, while a pressure generated from the operation of the regulator (e.g. either a first pump or an air valve) “pulls” on the moveable member. In terms of air pressure, the moveable member is affected on one side by the air pressure in the neck stoma generated naturally during respiration, and on the other side by the air pressure in the oral cavity that is simulated artificially using the regulator. The resulting airflow, or air pressure gradient, experienced by the moveable member causes it to vibrate and generate a voice.

In some examples, implementing the regulator as an air valve may facilitate miniaturisation and electronic power consumption of voice generation system 400 or 500 and improve user experience since an air valve may be quieter than an air pump during operation.

Voice generation systems 400 and 500 may further comprise, a processing system including voice enhancement software/hardware (in housing 410 or 510 following transducer module or in housing 450 or the denture unit 550 preceding the speaker module) to improve the quality or loudness of the voice generated by the voice generation system. In some examples, the processing system comprises an AI voice conversion module.

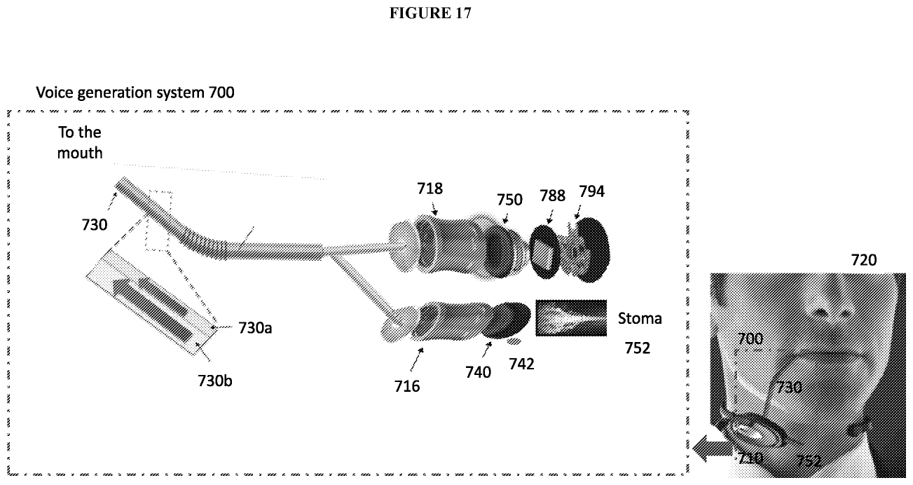

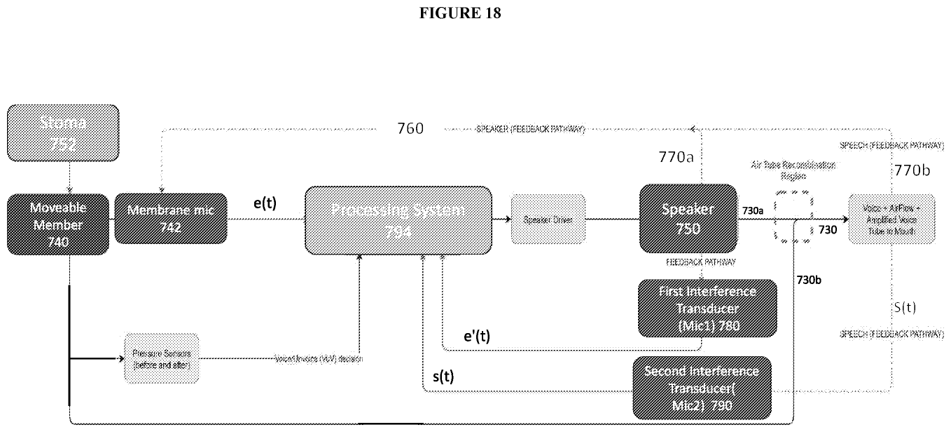

Referring to FIGS. 17-18, there is illustrated another example voice generation system 700.

As shown in FIG. 17, voice generation system 700 is the similar or same as the voice generation system 200, except voice generation system 700 will be used to describe by way of example how some embodiments of the voice generation system may be configured to reduce sound interference.

Referring to FIG. 17, the airflow and the sound are generated in two different sections or compartments of housing 710. Housing 710 comprises a first compartment 716 for generating the airflow and a second compartment 718 for synthesising the voice. First compartment 716 contains moveable member 740, the transducer module 742, and is in communication with the user's neck stoma 752, while second compartment 718 contains speaker module 750, as well as electronics for the operation of voice generation system 700, such as a battery 788 and a voice enhancement and/or conversion unit 792 (including hardware and software) comprising a processing system 794. The processing system 794 may be used to process the electrical signal generated by the transducer module 742. The processing system 794 may be used for processing the electrical signal adjusting the sound quality produced by speaker module 750. Additionally or alternatively, the processing system 794 may be configured to reduce sound interference received at the transducer module 742, which will be described in more detail next.

Compartments 716 and 718 may be coupled or linked so as to be in communication with tube 730. In this way, the airflow, moveable member vibration/sound and the speaker module sound can be combined and such that both travel into the oral cavity of user 720 along the same air passage. However, in some examples, it is desirable to reduce the sound interference 760 received at the transducer module 742. The sound interference 760 can be caused by the output of the speaker module sound 770a travelling from second compartment 718 into first compartment 716. The sound interference 760 is also caused by speech 770b travelling down from tube 730 into first compartment 716. The speech 770b is generated as a result of sound (from the tube 730) exiting the vocal tract to become modulated by the user's mouth or lips.

In such situations, it is desirable for the voice generation system 700 to have a processing system 794 configured to reduce sound interference received at the transducer module. The processing system 794 can be configured to reduce sound interference 760 based on at least one electrical signal received from the transducer module 742 that is configured to convert vibrations of the moveable member 740. Additionally, or alternatively, the processing system 794 can be configured to reduce sound interference 760 based on at least one electrical signal received from at least one interference transducer module for receiving sound interference 760, which could be interference transducer module 780 and/or interference transducer module 790. A further explanation is provided with reference to FIG. 18.

In some embodiments as shown in FIG. 18, the stoma airflow drives a moveable member 740, which in this example is a membrane, to generate sound e(t). e(t) is transferred to the oral cavity via a tube (the membrane tube 730b). The transducer module 742, which in this example is a microphone, is placed in the proximity of the membrane. The microphone picks the membrane sound e(t) and passes that to a speaker module 750, which in this example is a loudspeaker. The loudspeaker amplifies the sound and sends e(t) to the oral cavity via a second tube (the speaker tube 730a). The speaker and membrane tubes merge at some point to oral tube 730 and send the sound and airflow to the oral cavity.

The problem of these tubes merging to oral cavity is the speaker sound may find its way back via the tube 730 to reach to the membrane tube 730b via the oral cavity as acoustic feedback e′(t). This is specifically problematic when the speaker becomes louder or the membrane becomes quieter in the transition between voiced and unvoiced sounds.

If the patient intends to speak louder, increasing the speaker gain amplifies e′(t) compared to e(t) and the microphone starts picking up e′(t) instead of e(t), causing a positive feedback loop between microphone and the speaker.

Same problem exists in the voiced/unvoiced transitions of the voice generation system. The membrane generates voice automatically in voiced sounds and it becomes quiet in unvoiced sounds (driven by patient's mouth and stoma pressure variations). When membrane is generating voice, the microphone normally hears membrane voice e(t) because of the proximity to the membrane. As the membrane starts to become quiet in voiced to unvoiced transitions, the membrane sound e(t) fades to zero. However, the speaker sound e′(t) inside the oral cavity does not fade simultaneously. The oral cavity is a resonant cavity which means that it preserves the speaker sound e′(t) inside it longer. Hence, the membrane microphone starts picking the e′(t) and an acoustic echo or feedback loop is shaped in the voiced/unvoiced transitions.

The other sound interference problem may be occurred in this system is the speech signal s(t) that the mouth is naturally generating using the voice generation system. Embodiments of the voice generation system described in this specification relate to the membrane and speaker sounds e(t)+e′(t) to excite the mouth and generate speech signal (s(t)) which is naturally generated by the user as they move their face/lips muscles. In a similar scenario in addition to speaker sound e′(t), the speech signal s(t) also finds its path to reach the membrane microphone as an unwanted interference.

If there is no sufficient acoustic feedback reduction (e.g. suppression) is in place in the system, the feedback/noise signals e′(t)+s(t) will dilute the membrane microphone signal and shape a positive feedback loop.

As explained above there are two feedback paths identifiable in our system. A feedback reduction module identifies and measures these paths and removes them from the membrane mic signal in real-time. These feedback paths are:
1. The speaker feedback voice (e′(t)). This can be measured by a first interference transducer module 780, which in this example is a microphone, referred to as “mic1” placed at the speaker tube (mic1) close to the mouth, and
2. The speech signal s(t) feedback. This can be measured by a second interference transducer module 790, which in this example is an ambient microphone, referred to as “mic2”, such that the microphone is exposed to ambient and can monitor the airborne speech signal as the speech signal s(t) propagates outside the mouth.

The feedback reduction system monitors the speaker feedback signal (e′(t)) and the speech interference signal s(t) in real-time. Then it uses either or both of the two main approaches to remove the unwanted feedback (noise):
1) forward suppression
2) adaptive feedback cancellation or residual feedback reduction approach.

The forward suppression approach aims to prevent the feedback from happening in the first place. The adaptive feedback cancellations or residual feedback reduction tend to reduce the feedback after it is present.

Notable examples of forward suppression applicable in this system include automatic gain control including frequency selective gain control (limiting speaker gain automatically and at certain frequencies that relate to feedback paths to avoid feedback), phase modulation of speaker and mic or frequency shifting to make it easier for the membrane mic to differentiate between e(t) and e′(t)).

These examples of forward suppression may be less useful for louder voices or reduce the speech signal. So some embodiments may benefit from method adaptive feedback cancellation or residual feedback suppression. The main methods for adaptive feedback cancellation is adaptive filtering or using AI algorithms that learn to discriminate between the membrane sound e(t) and speech signal s(t).

In some embodiments a normalized least-mean-squares (NLMS) algorithm is used for adaptive feedback cancellation which is fast enough for real-time application. These adaptive feedback methods use the microphone signals (mic 1 780 and mic 2 790) to recursively approximate the feedback path and remove acoustic feedback signals the membrane mic signal. They work by minimizing an error signal between e(t)+echo and e(t) while the echo is (e′(t)+s(t) provided by the microphones (mic1 780 and mic 2 790).

Mic 1 and “membrane microphone” also are used in some embodiments to assist this interference reduction approach further to estimate the transfer function of the tube 730 which further modifies the speaker feedback e′(t) when it travels back via the tube to reach the membrane mic.

The feedback reduction in the voice generation system is further strengthened by the voiced unvoiced decision. This helps feedback elimination in voiced/unvoiced transitions as the voice generation system turns the mic off unvoiced sounds in real-time. The use of voiced/unvoiced decision for feedback reduction has been mentioned earlier in the detailed description.

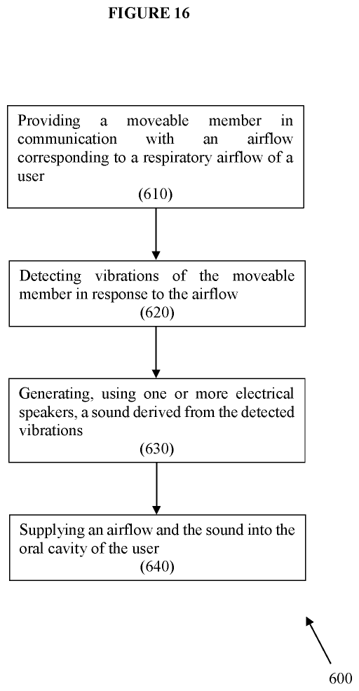

Referring to FIG. 16, there is illustrated a method 600 of generating a voice. Method 600 comprises step 610 of providing a moveable member in communication with an airflow corresponding to a respiratory airflow of a user. Then, method 600 comprises step 620 of detecting vibrations of the moveable member in response to the airflow. Then, method 600 comprises step 630 of generating, using one or more electrical speakers, a sound derived from the detected vibrations. Then, method 600 comprises step 640 of supplying an airflow and the sound into the oral cavity of the user.

In some examples, the airflow supplied in step 640 is the same airflow that is in communication with the moveable member (e.g. the respiratory airflow). In other examples, the airflow supplied in step 640 is a different airflow than that in communication with the moveable member.

In some examples, the moveable member is provided in an air channel which is subject to a first air pressure at a first end of the air channel and a second air pressure at a second end of the air channel. In some examples, the first air pressure corresponds to an air pressure of a neck stoma of the user, and wherein the second air pressure corresponds to an air pressure in the oral cavity of the user.

In some examples, method 600 further comprises sensing the air pressure and/or an airflow (i.e. the respiratory airflow) at the neck stoma of the user, and generating, using an air pump, the first air pressure and the airflow (i.e. the respiratory airflow) of the neck stoma. In some examples, method 600 further comprises sensing the air pressure in the oral cavity of the user, and generating, using an airflow control element or regulator, the second air pressure. In some examples, the airflow control element is an air pump. In some examples, the airflow control element is an air valve.

In some examples, method 600 further comprises channelling or guiding, within an air passage, a respiratory airflow of the user, wherein the airflow is the channelled respiratory airflow.

In some examples, method 600 further comprises converting, using a transducer, the vibrations of the moveable member into an electrical signal. Method 600 may further comprise processing this electrical signal (representing the voice), before supplying the processed electrical signal to the one or more electrical speakers for generating the sound. The step of processing the electrical signal may comprise improving or enhancing a volume (e.g. increasing the loudness) or quality of the voice encoded or represented by the electrical signal. The processing step may include a voice-conversion software/hardware based on AI to improve acoustic features or naturalness of the voice.

In some examples, method 600 further comprises reducing sound interference generated by the one or more electrical speakers.

Optional embodiments may also be said to broadly include the parts, elements, steps and/or features referred to or indicated herein, individually or in any combination of two or more of the parts, elements, steps and/or features, and wherein specific integers are mentioned which have known equivalents in the art to which the invention relates, such known equivalents are deemed to be incorporated herein as if individually set forth.

Although a preferred embodiment has been described in detail, it should be understood that many modifications, changes, substitutions or alterations will be apparent to those skilled in the art without departing from the scope of the present invention.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

### Disclosed Features

1. A voice generation system comprising:

a housing comprising a first opening and a second opening, the housing defining an air passage between the first opening and the second opening;

a moveable member located within the housing and configured to vibrate in response to air flowing in the air passage;

a transducer module configured to convert vibrations of the moveable member into an electrical signal; and

a speaker module configured to convert the electrical signal into sound and to output the sound into the oral cavity of a user.
2. The voice generation system of clause 1, wherein the air flowing in the air passage is due to an air pressure difference between the first opening and the second opening.
3. The voice generation system of clause 1 or 2, wherein the first opening is configured to be in communication with a neck stoma of the user.
4. The voice generation system of clause 3, wherein the housing is configured to attach to the neck or chest of the user such that the first opening is in connection with the neck stoma.
5. The voice generation system of any one of clauses 1 to 4, wherein the second opening is configured to be in communication with the oral cavity of the user.
6. The voice generation system of any one of clauses 1 to 5, further comprising a tube having a first end connected to the housing around the second opening, wherein a second end of the tube is configured to be inserted into the oral cavity of the user.
7. The voice generation system of clause 6, wherein the housing and the tube together define an air passage between the first opening of the housing and the second end of the tube.
8. The voice generation system of any one of clauses 1 to 7, wherein the speaker module is located within the housing, and wherein the speaker module is configured to output the sound through the second opening.
9. The voice generation system of clause 1, wherein the housing is configured to connect to an airflow source, the airflow source being configured to generate an airflow and to output the airflow into the oral cavity of the user.
10. The voice generation system of clause 9, wherein the airflow source is a neck stoma of the user and the airflow is a respiratory airflow outputted from the user through the neck stoma.
11. The voice generation system of clause 9, wherein the airflow source is an air pump.
12. The voice generation system of clause 1, further comprising:

a pressure sensing module configured to attach to the neck of the user and to sense an air pressure at a neck stoma of the user;

an air pump located within the housing and configured to generate an airflow that moves along the air passage from the first opening to the second opening; and

a controller configured to control the air pump based on the sensed air pressure at the neck stoma.
13. The voice generation system of clause 12, wherein the air flowing in the air passage is due to a difference in the air pressure in the air passage and the air pressure at the second opening.
14. The voice generation system of clause 12 or 13, wherein the second opening is configured to be in communication with the oral cavity of the user.
15. The voice generation system of any one of clauses 12 to 14, further comprising a tube having a first end connected to the housing around the second opening, wherein a second end of the tube is configured to be inserted into the oral cavity of the user.
16. The voice generation system of clause 15, wherein the housing and the tube together define an air passage between the first opening of the housing and the second end of the tube.
17. The voice generation system of any one of clauses 12 to 16, wherein the speaker module is located within the housing, and wherein the speaker module is configured to output the sound through the second opening.
18. The voice generation system of any one of clauses 12 to 17, wherein the air pump is configured to generate an air pressure in the air passage corresponding to the sensed air pressure at the neck stoma.
19. The voice generation system of any one of clauses 12 to 18, wherein the housing is configured to be secured to an auricle of the user.

1. A voice generation system comprising:
a housing comprising a first opening and a second opening, the housing defining an air passage between the first opening and the second opening;
a moveable member located within the housing and configured to vibrate in response to air flowing in the air passage;
a transducer module configured to convert vibrations of the moveable member into an electrical signal; and
a speaker module configured to convert the electrical signal into sound and to output sound into the oral cavity of a user;
wherein, the voice generation system is configured such that during use, the air pressure at or near the second opening corresponds to an air pressure in the oral cavity of the user.

2.-74. (canceled)
